(19) United States
(12) Patent Application Publication
(10) Pub. No.: US 2014/0213757 A1
(43) Pub. Date: Jul. 31, 2014

(54) CASSETTE FOR RADIOPHARMACEUTICAL SYNTHESIS

(71) Applicant: GE HEALTHCARE LIMITED, BUCKINGHAMSHIRE (GB)

(72) Inventors: Jonathan R Shales, Buckinghamshire (GB); Roger P Pettitt, Bucks (GB)

(21) Appl. No.: 14/343,139

(22) PCT Filed: Sep. 18, 2012
(86) PCT No.: PCT/US2012/055863

§ 371 Date: Mar. 6, 2014

Related U.S. Application Data
(60) Provisional application No. 61/541,178, filed on Sep. 30, 2011.

(51) Int. Cl. A61K 51/08

(52) U.S. Cl. CPC A61K 51/088
USPC 530/317; 422/554

(57) ABSTRACT

The present invention is directed to a modified synthesis cassette (110) that enables flexible, in-process monitoring of radiopharmaceutical synthesis. Also provided is a method for radiopharmaceutical synthesis using the modified synthesis cassette (110).

### FIELD OF THE INVENTION

The present invention is directed to the field of radiopharmaceutical synthesis. More specifically, the present invention is directed to a modified synthesis cassette that enables flexible, in-process monitoring of radiopharmaceutical synthesis and a method for using same.

### BACKGROUND OF THE INVENTION

Radiopharmaceuticals, or radiotracer, can be synthesized using automated synthesis platforms using specially-tailored cassettes. For example, the synthesis of Fluciclatide [¹⁸F] Injection, a PET agent for imaging malignant diseases, can be performed using either the TRACERlab FX F-N platform or the FASTlab™ platform, both sold by GE Healthcare, Liege, Belgium. The use of specially-tailored, single-use cassettes (e.g., the FASTlab™ cassette) is widely accepted for its convenience and for its ability to confine any radioactive waste to the cassette alone.

Commercial PET production facilities are often set up solely for the production of a single radiotracer (e.g., ¹⁸F-FDG). However, as other radiotracers are developed and adopted, the production facilities will need to be able to produce these other radiotracers as well. The FASTlab™ system was designed from the start as a multi-tracer platform so as to enable a given production facility to offer multiple radiotracers without requiring costly expansion of the production areas. The FASTlab™ system comprises a synthesis unit which operates a single-use cassette removable mounted thereon. The spent cassette is removed after the synthesis run and replaced by a fresh cassette which may be likewise operated to perform a synthesis run. Cassettes may be tailored to produce a specific radiotracer, and the synthesis unit is programmed to operate each different type of cassette to synthesize its particular tracer.

One short-coming of the current automated synthesis platforms for radiopharmaceuticals is that all but one of the radiodetectors are fixed by the system and cannot be easily moved to different positions along the synthesis cassette. As the platforms should accommodate more than one tracer, and as there is a need for real-time monitoring (especially during product development and QC), there is therefore a need for means to increase the flexibility of the current automated synthesis platforms, to enable real-time monitoring of radio activity for the synthesis of a variety of different radiotracers.

### SUMMARY OF THE INVENTION

In view of the needs of the art, the present invention provides a synthesis cassette that enables flexible, in-process monitoring of radiopharmaceutical synthesis. The present invention also provides a kit including the synthesis cassette, an automated synthesis system incorporating the cassette, as well as a method for radiopharmaceutical synthesis using the synthesis cassette.

### BRIEF DESCRIPTION OF THE DRAWINGS

FIG. 1 shows a prior art cassette for the production of Fluciclatide (18F) Injection, showing the fluid paths, prefilled reagents and the SPE separation cartridges.

FIG. 2 is an alternative view of the cassette shown in FIG. 1 depicting the components connected to the cassette prior to synthesis.

FIG. 3 shows a method of monitoring the tC2 cartridges with an unshielded detector removably attached to the front of the prior art cassette.

FIG. 4 shows cassette cover of the present invention with a detector and shield mounted on the cassette cover.

FIG. 5 shows an alternative embodiment of the cassette cover of the present invention with the detector and shield mounted on the cassette cover.

FIG. 6 shows traces from unshielded radio-detector monitoring both purification cartridges (tC2 cartridges) and shielded radio-detector monitoring a single purification cartridge, displaying improved sensitivity/signal definition.

FIG. 7 shows two traces and the corresponding movements of syringe driver according to an example of the invention.

FIG. 8 shows two traces and the corresponding movements of syringe driver according to another example of the invention.

### DETAILED DESCRIPTION OF THE INVENTION

In order to develop an optimum and robust purification process there are three key parameters to consider and a number of factors affect each of these parameters.
Trapping. The crude radiopharmaceutical has to be transferred to the purification cartridges and retained whilst allowing excess liquid and impurities to pass through to waste.
Purification. The crude product should be retained on the cartridges whilst chemical and radioactivity impurities are removed and sent to waste by passing a purification solution through the cartridges.
Elution. Once purification has taken place, then the pure product has to be eluted from the cartridges and collected.

Each of these steps should be optimised and robust. Very often the result is a compromise. For example, washing the cartridges too vigorously removes all of the impurities, however, it will also remove some of the pure product and the Radio Chemical Yield (RCY) will be adversely affected. Conversely, too little washing results in a higher RCY but also a higher concentration of undesirable impurities.

Each of the Trapping, Purification and Elution processes can be affected by a number of variables such as pH, solvent concentration, temperature, pressure, vacuum, flow rate, etc. When optimizing each step it is difficult to monitor exactly what effect each change has had. A traditional way to evaluate each modification to the process would be to slow or stop the process and collect samples for analysis from the waste from the cartridges. Each fraction collected can be analysed, for example by HPLC or by measuring the radioactivity in an ion chamber, and a picture of what is happening can be built up. However, by interrupting the process artefacts can be introduced that would not normally be present, not to mention that the fraction collection process can be time consuming and also there is radioactivity exposer to the operator.

One embodiment of the invention provides a radiopharmaceutical synthesis cassette which enables the use of a user configurable radiodetector which can monitor radioactivity in any position along the cassette. This modified cassette offers many advantages in the development of novel tracers for an automated radiopharmaceutical synthesis platform. The modified cassette also enables the real time monitoring of the synthesis of more than one tracer for a given platform, and thus improved the quality control of the radiopharmaceutical production.

### The Synthesis Cassette and Device

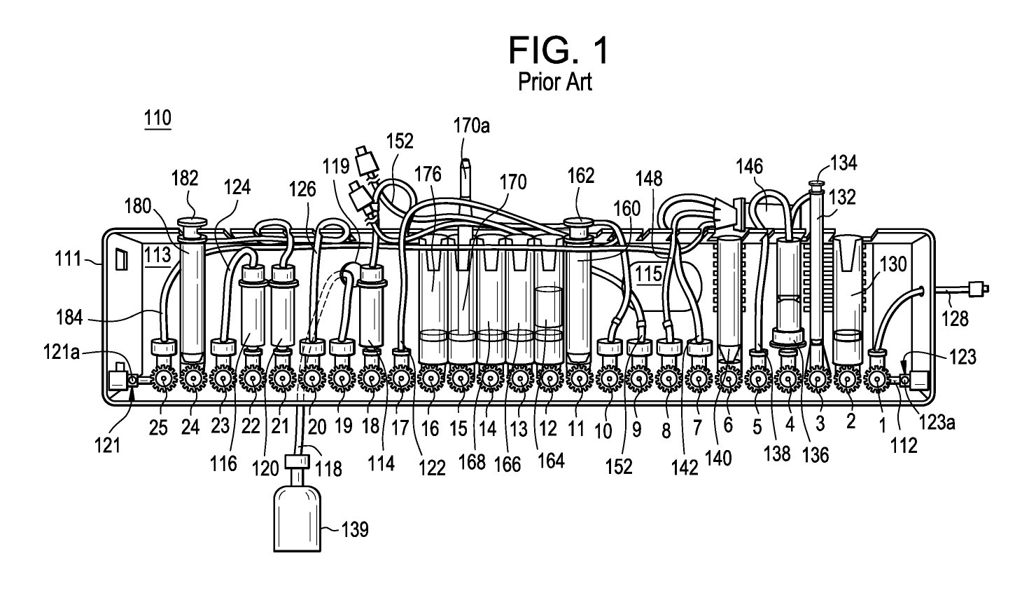

Reference is now made to FIG. 1, which depicts a disposable synthesis cassette 110 and its components. Cassette 110 includes, a manifold 112 including twenty-five 3 way/3 position stopcocks valves 1-25, respectively. Manifold valves 1-25 are also referred to as their manifold positions 1-25 respectively. Manifold valves 1, 4-5, 7-10, 17-23, and 25 have female luer connectors projecting up therefrom. Valves 2, 6, and 12-16 have an elongate open vial housing upstanding therefrom and support an upstanding cannula therein for piercing a reagent vial inserted in the respective vial housing. Movement of the reagent vial to be pierced by the respective cannula is performed under actuation by the synthesizer device. Valves 3, 11, and 24 support an elongate open syringe barrel upstanding therefrom. Valves 1-25 include three open ports opening to adjacent manifold valves and to their respective luer connectors, cannulas, and syringe barrels. Each valve includes a rotatable stopcock which puts any two of the three associated ports in fluid communication with each other while fluidically isolating the third port. Manifold 112 further includes, at opposing ends thereof, first and second socket connectors 121 and 123, each defining ports 121a and 123a, respectively. Manifold 112 and the stopcocks of valves 1-25 are desirably formed from a polymeric material, e.g. PP, PE, Polysulfone, Ultem, or Peek.

Cassette 110 is a variant of a pre-assembled unit designed to be adaptable for synthesizing clinical batches of different radiopharmaceuticals with minimal customer installation and connections. Cassette 110 includes reaction chamber/vessel, reagent vials, cartridges, filters, syringes, tubings, and connectors for synthesizing a radiotracer. Connections are desirably automatically made to the reagent vials by driving the septums thereof onto penetrating spikes to allow the synthesizer access to the reagents.

Cassette 110 is attachable to a synthesis device, such as FASTlab, which cooperatively engages the cassette so as to be able to actuate each of the stopcocks and syringes to drive a source fluid with a radioisotope through the cassette for performance of a chemical synthesis process. Additionally, the synthesis device can provide heat to the reaction vessel of cassette 110 as required for chemical reactions. The synthesizer is programmed to operate pumps, syringes, valves, heating element, and controls the provision of nitrogen and application of vacuum to the cassette so as to direct the source fluid into mixing with the reagents, performing the chemical reactions, through the appropriate purification cartridges, and selectively pumping the output tracer and waste fluids into appropriate vial receptacles outside the cassette. The fluid collected in the output vial is typically input into another system for either purification and/or dispensement. After product dispensement, the internal components of cassette 110 are typically flushed to remove latent radioactivity from the cassette, although some activity will remain. Cassette 110 thus can be operated to perform a two-step radiosynthesis process. By incorporating SPE cartridges on the manifold, cassette 110 is further able to provide simple purification so as to obviate the need for HPLC.

### The Cassette Setup for Synthesis of a Radiopharmaceutical—Fluciclatide (¹⁸F)

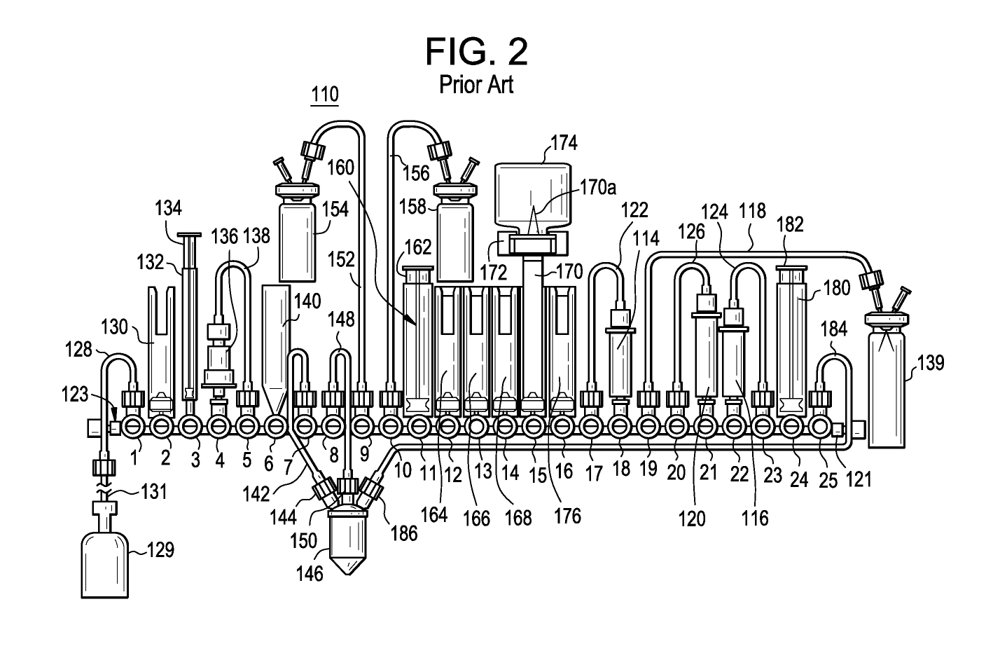

FIG. 1 further depicts a fully assembled cassette 110 for the production of Fluciclatide (¹⁸F) Injection, showing all tubing and prefilled reagent vials. While the cassette for producing Fluciclatide (¹⁸F) Injection is shown and described, the present invention is not limited to such a cassette or tracer and is contemplated to be suitable for any combination of cassette and purification cartridge for which it may be adapted. Cassette 110 includes a polymeric housing 111 having a planar major front surface 113 and defining a housing cavity 115 in which manifold 112 is supported. A first reverse phase SPE Cartridge 114 is positioned at manifold position 18 while a second reverse phase SPE cartridge 116 is positioned at manifold position 22. A normal phase (or amino) SPE cartridge 120 is located at manifold position 21. First SPE Cartridge 114 is used for primary purification. The amino cartridge 120 is used for secondary purification. The second SPE Cartridge 116 is used for solvent exchange. A Tygon tubing 118 is connected between cassette position 19 and a product collection vial 139 in which collects the formulation of the drug substance. Tubing 118 is shown in partial phantom line to indicate where is passing behind front surface 113 on the far side of manifold 112 in the view. While some of the tubings of the cassette are, or will be, identified as being made from a specific material, the tubings employed in cassette 110 may be formed from any suitable polymer and may be of any length as required. Surface 113 of housing 111 defines an aperture 119 through which tubing 118 transits between valve 19 and the product collection vial 139. FIG. 2 depicts the same assembled manifold of the cassette and shows the connections to a vial containing a mixture of 40% MeCN and 60% water at manifold position 9, a vial of 100% MeCN at manifold position 10, a water vial connected at the spike of manifold position 15, and a product collection vial connected at manifold position 19. FIG. 2 depicts manifold 112 from the opposite face, such that the rotatable stopcocks and the ports 121a and 123a are hidden from view.

Tubing 122 extends between the free end of cartridge 114 and the luer connector of manifold valve 17. Tubing 124 extends between the free end of cartridge 116 and the luer connector of manifold valve 23. Tubing 126 extends between the free end of cartridge 120 and the luer connector of manifold valve 20. Additionally, tubing 128 extends from the luer connector of manifold valve 1 to a target recovery vessel 129 (shown in FIG. 2) which recovers the waste enriched water after the fluoride has been removed by the QMA cartridge. The free end of tubing 128 supports a connector 131, such as a luer fitting or an elongate needle and associated tubing, for connecting the cavity to the target recovery vessel 129. In the method, the radioisotope is [¹⁸F]fluoride provided in solution with H₂[¹⁸O] target water and is introduced at manifold valve 6.

A tetrabutylammonium bicarbonate eluent vial 130 is positioned within the vial housing at manifold valve 2 and is to be impaled on the spike therein. An elongate 1 mL syringe pump 132 is positioned at manifold valve 3. Syringe pump 132 includes an elongate piston rod 134 which is reciprocally moveable by the synthesis device to draw and pump fluid through manifold 112 and the attached components. QMA cartridge 136 is supported on the luer connector of manifold valve 4 and is connected via silicone tubing 138 to the luer connector of manifold position 5. Cartridge 136 is desirably a QMA light carbonate cartridge sold by Waters, a division of Millipore. The tetrabutylammonium bicarbonate in an 80% acetonitrile: 20% water (v/v) solution provides elution of [¹⁸F]fluoride from QMA and phase transfer catalyst. A fluoride inlet reservoir 140 is supported at manifold valve 6.

Manifold valve 7 supports tubing 142 at its luer connector which extends to a first port 144 of a reaction vessel 146. The luer connector of manifold valve 8 is connected via a length of tubing 148 to a second port 150 of reaction vessel 146. The luer connector of manifold valve 9 is connected via tubing 152 to a vial 154 containing a mixture of 40% MeCN and 60% water (v/v). The acetonitrile and water mixture is used to enable primary purification of fluciclatide at the first SPE cartridge 114. The luer connector of manifold valve 10 is connected via tubing 156 to a vial 158 containing 100% MeCN used for conditioning of the cartridges and the elution of fluciclatide from the first SPE cartridge 114. Manifold valve 11 supports a barrel wall for a 5 ml syringe pump 160. Syringe pump 160 includes an elongate piston rod 162 which is reciprocally moveable by the synthesis device so as to draw and pump fluid through manifold 112. The vial housing at manifold valve 12 receives vial 164 containing 6-ethoxymethoxy-2-(4′-(N-formyl-N-methyl)amino-3′-nitro)phenylbenzothiazole). The vial housing at manifold valve 13 receives a vial 166 containing 4M hydrochloric acid. The hydrochloric acid provides deprotection of the radiolabelled intermediate. The vial housing at manifold valve 14 receives a vial 168 of a methanol solution of sodium methoxide. The vial housing at manifold valve 15 receives an elongate hollow spike extension 170 which is positioned over the cannula at manifold valve 15 and provides an elongate water bag spike 170a at the free end thereof. Spike 170 pierces a cap 172 of water bottle 174 containing water for both diluting and rinsing the fluid flowpaths of cassette 110. The vial housing at manifold valve 16 receives a vial 176 containing ethanol. Ethanol is used for the elution of the drug substance from the second SPE cartridge 116. The luer connector of manifold valve 17 is connected to silicone tubing 122 to SPE cartridge 114 at position 18. Manifold valve 24 supports the elongate barrel of a 5 ml syringe pump 180. Syringe pump 180 includes an elongate syringe rod 182 which is reciprocally moveable by the synthesis device to draw and pump fluid through manifold 112 and the attached components. The luer connector of manifold valve 25 is connected to tubing 184 to a third port 186 of reactor vessel 146.

Cassette 110 is mated to an automated synthesizer having rotatable arms which engage each of the stopcocks of valves 1-25 and can position each in a desired orientation throughout cassette operation. The synthesizer also includes a pair of spigots, one of each of which insert into ports 121a and 123a of connectors 121 and 123 in fluid-tight connection. The two spigots respectively provide a source of nitrogen and a vacuum to manifold 112 so as to assist in fluid transfer therethrough and to operate cassette 110. The free ends of the syringe plungers are engaged by cooperating members from the synthesizer, which will then apply the reciprocating motion thereto within the syringes. A bottle containing water is fitted to the synthesizer then pressed onto spike 170 to provide access to a fluid for driving compounds under operation of the various-included syringes. The reaction vessel will be emplaced within the reaction well of the synthesizer and the product collection vial, waste vial, and source reservoir are connected.

The synthesizer includes a radioisotope delivery conduit which extends from a source of the radioisotope, typically either vial or the output line from a cyclotron, to a delivery plunger. The delivery plunger is moveable by the synthesizer from a first raised position allowing the cassette to be attached to the synthesizer, to a second lowered position where the plunger is inserted into the housing at manifold valve 6. The plunger provides sealed engagement with the housing at manifold valve 6 so that the vacuum applied by the synthesizer to manifold 112 will draw the radioisotope through the radioisotope delivery conduit and into manifold 112 for processing. Additionally, prior to beginning the synthesis process, arms from the synthesizer will press the reagent vials onto the cannulas of manifold 112. The synthesis process may then commence.

Some of the 25 manifold positions of the cassette are predefined, for example the three syringes, and cannot be configured to different positions, and some positions, for example 7-10 and 16-23, can be defined by the user depending on the requirements for a particular tracer. Therefore, the cassette layout for new tracers can differ from an FDG cassette and from other novel tracer cassettes.

### The Cassette and Related Aspects of the Present Invention

The FASTlab™ synthesiser is configured with four on-board radiodetectors that are used to monitor the synthesis of FDG and the option of placing a single external detector against the cassette (which connects to the synthesizer at a port at the rear of the FASTlab). The detectors monitor the incoming activity on the QMA cartridge at manifold position 4 of the cassette, and the activity at the reactor vessel, at the purification cartridge at manifold position 18 and at the syringe at manifold position 24. With the standard on-board FDG detector configuration it is only possible to monitor the positions as detailed here.

For the development of novel tracers it is often desirable to monitor radioactivity at different positions on the cassette. For example, the purification of the crude Fluciclatide product takes place on two Solid Phase Extraction (SPE) cartridges at positions #20 and #22. Thus, the use of a cassette which enables a radiodetector or detectors to be focussed on one or both of the purification cartridges can provide real time information on how radioactivity is being trapped, purified and eluted from the cartridges without introducing artefacts to the process by interrupting the process and without the operator receiving any extra personal dose. The information received can be used to modify and optimise conditions for the three key steps, saving time, resource and reducing operator exposure. Furthermore, the use of such modified cassettes also provides flexibility such that the synthesis process for different radiopharmaceuticals can be monitored without the need to reconfigure the synthesis device.

Thus, one aspect of the invention provides a cassette for synthesizing a radiopharmaceutical, comprising an elongate manifold including multiple stopcock positions each connectable among a reaction chamber, tubings, and at least one separations cartridge used in synthesizing the radiopharmaceutical; and a cassette housing supporting the manifold therein, which housing comprises an elongate planar base wall supporting a transversely-oriented perimetrical wall thereabout; wherein the housing comprises means for securing one or more connectors, each said connector being adapted to receive a radiodetector at a location of the housing such that the radiodetector is capable of detecting radioactivity at a single stopcock position. The connector may comprise a substrate formed from a radiation-shielding material defining an aperture therethrough that is placed in registry with the desired location on the manifold.

The connector on of the housing can take many forms.

Thus, in one embodiment, the housing, for example on the planar face thereof, could include receptacles through which the radiation shield is secured by wedging, screwing, bolting or nailing.

Alternatively, in another embodiment, the housing, for example on the planar face thereof, could include receptacles for securing the radiation shield through plugging.

In still another embodiment, the housing, for example on the planar face thereof, could also include receptacles for securing the radiation shield through a pair of magnets.

In one embodiment, the housing, for example on the planar face thereof, wall optionally further comprises means for securing the radiodetector.

The cassette of the present invention enables flexibility and quick configuration of a radiation shield and detector, allowing the monitoring of any position on the cassette.

The radiation shield of the connector can be any standard lead shield to provide shielding from other sources of radioactivity around the cassette. The radiation detector can be any standard detector, e.g., detector for PET applications. Preferred detectors are those have a compact size and also provide a suitable response range. An exemplary detector is the solid state PIN diode detector.

By using the shield, the radiation detector becomes directional and by moving the detector within the shield a collimator effect can be achieved. The shielded radio-detector provides much better sensitivity/signal definition when compared to an unshielded radio-detector taped to the front of the cassette (see Example below).

In another aspect of the invention, it is provided a kit for synthesizing a radiopharmaceutical. The kit comprises a cassette according to the first aspect of the invention, as well as means to secure the one or more radiation shield to the transversely-oriented perimetrical wall.

The means to secure the one or more shield can include a variety of mechanisms.

Thus, in one embodiment, the means to secure the one or more radiation shield to the housing includes wedges, screws, bolts or nails.

In another embodiment, means to secure the one or more radiation shield to the housing includes a pair of magnets.

In one embodiment, the kit further comprises one or more radiation shields.

In another embodiment, the kit further comprises one or more radiodetectors.

In still another aspect of the invention, it is provided an automated synthesis platform for radiopharmaceuticals including the cassette according to the first aspect of the invention and a synthesis unit.

A further aspect of the invention provides the use of the cassette according to the first aspect of the invention for synthesizing a radiopharmaceutical.

### EXAMPLES

The following examples illustrate the synthesis cassette according to certain embodiments of the invention, and the use of the cassette for monitoring the production process for a radiopharmaceutical. The cassette enables monitoring of the radioactivity on certain parts of the cassette that were not previously monitored by the synthesizer's on-board radio-detectors.

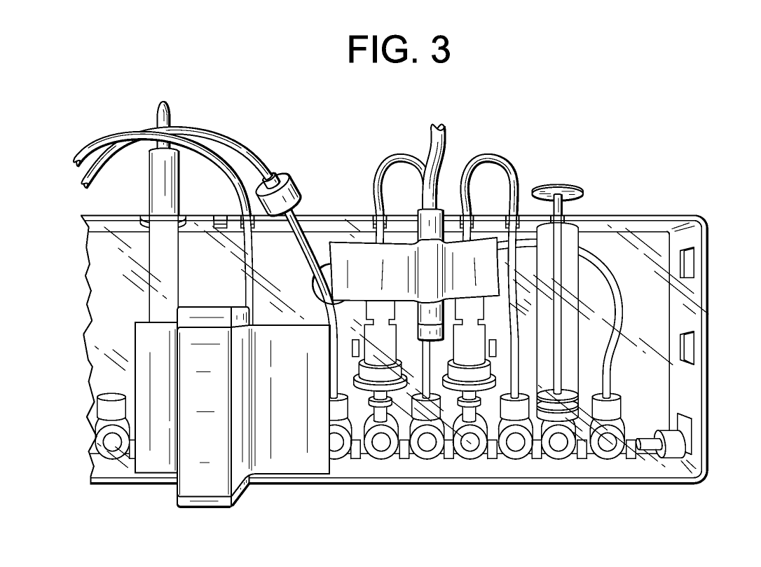

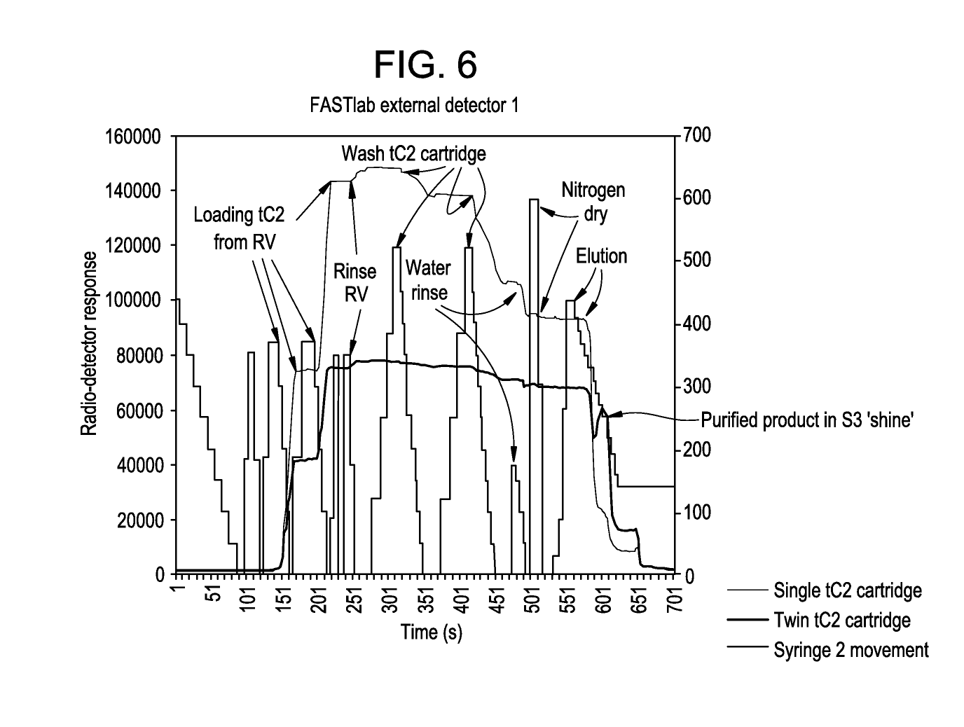

During the development of the solid phase extraction purification step for Fluciclatide, the radioactivity movements around the two purification cartridges was monitored by an external radiation detector (it is connected to the connector labeled ‘External Input 1’ at the rear of the FASTlab device). Initially, the radiation detector was taped to the front of the cassette between the two cartridges such that the detector is not shielded. (FIG. 3). (Since the unshielded detector is not directional or collimated, there was no point in trying to position the detector in front of either SPE cartridge). Therefore, in order to be broadly consistent from one synthesis to another, it was positioned approximately between the two SPE cartridges (an Illustrative detection graph is shown in FIG. 6, see the plot for the Twin tC2 cartridges). At around 600 seconds, a peak can be observed indicating the shine from purified product in S3 cartridge. FIG. 3 also shows a taped block to the left of the radiation detector, which is a tungsten syringe shield with some extra lead stuffed inside. Since the taped on radiation detector is not shielded it is susceptible to responding to any radioactive source not just the sources from the SPE cartridges. One of the main radioactive sources on the FASTlab is the reaction vessel (RV) which is positioned towards the front of the FASTlab and below the cassette on the left hand side. So the tungsten/lead block provides some shielding between the taped on detector and the reaction vessel.

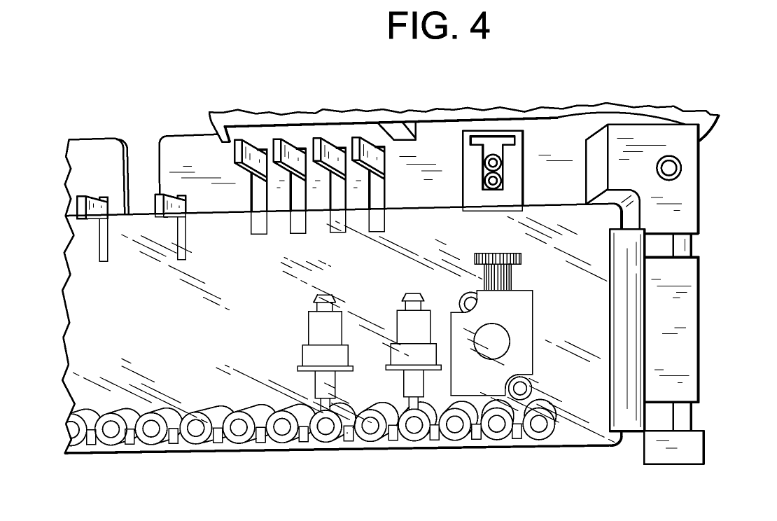

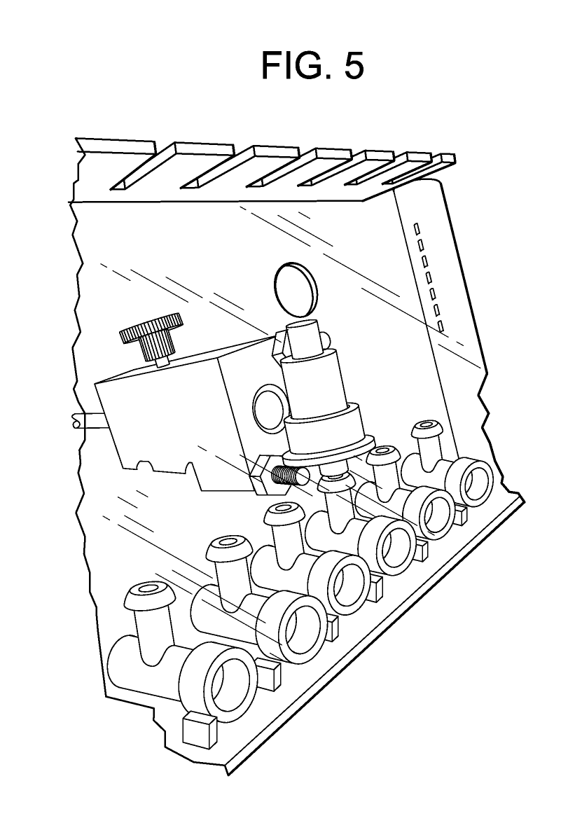

To eliminate the shine and provide flexibility and ease of attachment for the radiation shield and detector, receptacles were included on the planar face of the cassette, such that the radiation shield can be easily attached and detached. FIG. 4 shows a modified cassette on which a single radiation shield is attached through screws. An alternative view from the far side of the cassette is shown in FIG. 5. A detector is inserted into the shield, which directly faces a single cartridge in the cassette. Radiation detection by this detector set up is observed in FIG. 6 (see the plot for the Single tC2 cartridge). While the two loading events from the reaction vessel are clearly observed (around 150 and 200 seconds, respectively), no interference from adjacent cartridges (or shine) is visible.

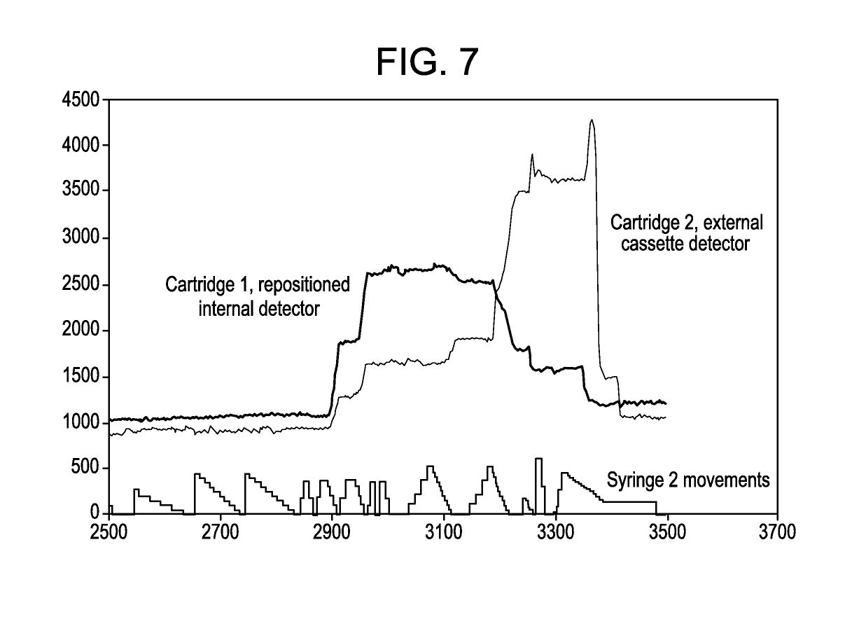

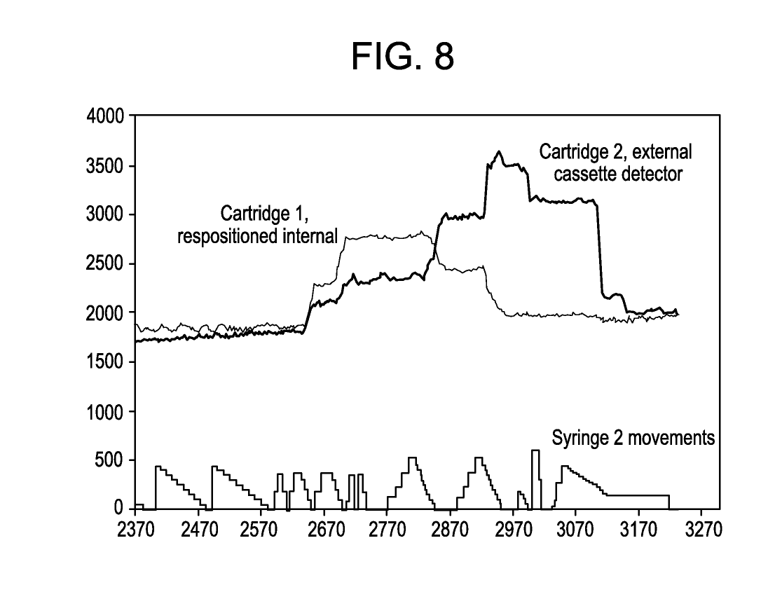

FIGS. 7 and 8 show two radioactive traces from another set of experiments, and the corresponding movements of syringe driver #2 (S2). During the purification of crude product by Solid Phase Extraction (SPE) cartridges, it is useful to overlay the S2 movements on the radioactive traces in order to be able to identify specific events in the radioactive trace. In this case S2 is used to transfer the crude product from the Reaction Vessel (RV) to the SPE cartridges. S2 is also used to pass the purifying solution and also the elution solution through the SPE cartridges. An increase in the response from S2 shows the plunger of S2 is being drawn up to increase the volume of S2 and vice versa.

The first SPE purification cartridge (here at position 20) is monitored by an internal detector that was repositioned from its usual position at #18 to position #20. This was done when the opportunity arose during a three year maintenance of a FASTlab and would not normally be undertaken by an operator without extensive training. The second SPE purification cartridge is monitored by the additional external detector attached to the outside of the cassette as described in this application.

In order to interpret correctly the two radioactive traces it is important to understand how the radioactivity is moving and presented to the detectors. Generally speaking, the radioactivity is moved from left to right through the cassette (See FIG. 2). The radioactivity enters the synthesis process at manifold position 6 of the cassette and waste products and contaminants are removed through manifold position 19 to vial 139. The movement of radioactivity can be by positive gas pressure, by vacuum or by the movement of one or more of the syringe drivers. An increase or decrease in the response from the detector usually corresponds to the movement of S2 where gas or liquid is being pushed through the cassette and SPE cartridges. Since S2 has a finite movement, equivalent to approximately 7 ml, an increase or decrease in response from the detector is usually followed by a static response or plateau as S2 is refilled ready for the next operation and this can be seen in the corresponding S2 movement trace.

The difference in maximum magnitude of response from the two detectors can be explained by a geometry effect. The internal detector is not positioned as close to the first cartridge as the external detector is to the second cartridge. Therefore, the internal detector will not respond as much to the same amount of radioactivity as the external detector will.

FIG. 7 shows a typical purification process after the process has been optimised. At approximately 2900 seconds the crude product is transferred from the reaction vessel to the two SPE cartridges in series (at valves 21 and 22). Although initially all of the radioactivity is presented to the first of these SPE cartridges and internal detector there is also a smaller response from the external detector. At approximately 3100 seconds, radioactivity can be seen to decrease in the first cartridge and increase in the second cartridge. At this stage, all the radioactivity is moving through the cartridges as the sample is being purified, however, the impurities are being washed away faster than the desired product, thus some of the radioactivity remains in the first cartridge whilst some is transferred to the second cartridge. Shortly before 3300 seconds there is a small but sudden drop in the response from the detector on the first cartridge and a corresponding small but sharp peak in the response from the detector on the second cartridge. This marks the end of the purification process where the last of the undesired impurities are removed from the first cartridge and pass through the second cartridge as shown by the decrease in response from the first cartridge detector and sharp peak from the second cartridge detector.

At this point the majority of the radioactivity being detected is due to the desired purified product and this is divided unequally between the two cartridges with the majority of the radioactivity located on the second cartridge. The next step in the process is to elute the purified product from the two cartridges. This can be observed just before 3400 seconds and is identified by a sudden drop in response from the detector on the first cartridge immediately followed by a sharp peak in response from the detector on the second cartridge. Then, as the desired purified product is collected in S3 further along the cassette to the right hand side, the response from the detector on the second cartridge reduces to a low level.

FIG. 8 shows undesirable conditions for the purification of the crude product. In this case the purification has been affected by increasing the temperature at which the process is performed and also increasing percentage of the organic component of the purification solution. The result of this is a much more aggressive purification where all of the undesired impurities are removed but also a significant amount of the desired purified product. This is observed at approximately 2900 seconds where the response from the detector on the first cartridge drops to almost background levels showing that all the radioactivity has been transferred to the second cartridge. This corresponds to a large response from the detector on the second cartridge followed by a drop in response at approximately 3000 seconds. In this example, there is no spike in the response of the detector on the second cartridge during the elution of the purified product at approximately 3100 seconds since there is no radioactivity left in the first cartridge to appear in front of the detector on the second cartridge. The traces from the detectors in this example show that the purification process is not optimized for maximum yield since it can be seen that some of the purified product has been removed and sent to waste. However, if only the purified end product were analyzed, the process may incorrectly be judged as successful since the analysis would only show the purity of the end product with no way to determine how much had been wasted.

While the particular embodiment of the present invention has been shown and described, it will be obvious to those skilled in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art.

What is claimed is:

1. A cassette for synthesizing a radiopharmaceutical, comprising:
an elongate manifold including multiple stopcock positions each connectable to a reaction chamber, tubings, and at least one separations cartridge used in synthesizing the radiopharmaceutical; and
a cassette housing supporting said manifold therein, said housing comprising an elongate planar base wall supporting a transversely-oriented perimetrical wall thereabout;
wherein the housing further comprises means for securing one or more radiation shields, each said radiation shield is adapted to receive a radiodetector at a location on the planar wall such that the radiodetector is capable of detecting radioactivity at a single stopcock position.

2. The cassette of claim 1, wherein the means for securing one or more radiation shield includes receptacles through which the radiation shield is secured by wedging, screwing, bolting or nailing.

3. The cassette of claim 1, wherein the means for securing one or more radiation shield includes receptacles for securing the radiation shield through plugging.

4. The cassette of claim 1, wherein the means for securing one or more radiation shield includes receptacles for securing the radiation shield through a pair of magnets.

5. The cassette of claim 1, wherein the planar base wall further comprises means for securing the radiodetector.

6. A kit for synthesizing a radiopharmaceutical, comprising the cassette of claim 1; and means to secure the one or more radiation shield to the housing.

7. The kit of claim 6, wherein the means to secure the one or more shield includes wedges, screws, bolts or nails.

8. The kit of claim 6, wherein the means to secure the one or more shield includes a pair of magnets, wherein one of the pair of magnets is physically engaged to the connector.

9. The kit of claim 6, further comprising one or more radiation shield.

10. The kit of claim 6, further comprising one or more radiodetectors.

11. An automated synthesis platform for radiopharmaceuticals including the cassette of claim 1 and a synthesis unit.

12. Use of the cassette of claim 1 for synthesizing a radiopharmaceutical.

13. The cassette of claim 1, further comprising one or more means for retentatively engaging one or more radiodetectors.

14. The cassette of claim 13, wherein said planar base wall further defines one or more apertures therethrough for engaging the connector.

15. The cassette of claim 13, wherein said planar base wall further includes one or more projections thereon for engaging the connector.

16. The cassette of claim 13, wherein said planar base wall further comprising one or more shelves for supporting the connector.
